SEQUENCE LISTING (3 sequences)

SEQ ID NO: 1 - protein, 2444 aa, Rat
MEEVVIAGMSGKLPESENLQEFWANLIGGVDMVTDDDRRWKAGLYGLPKRSGKLKDLSKFDASFFGVHPKQAHTMDPQLRLLLEVSYEAIVDGGINPASLRGTNTGVWVGVSGSEASEALSRDPETLLGYSMVGCQRAMMANRLSFFFDFKGPSIALDTACSSSLLALQNAYQAIRSGECPAATVGGINLLLKPNTSVQFMKLGMLSPDGTCRSFDDSGNGYCRAEAVVAVLLTKKSLARRVYATILNAGTNTDGCKEQGVTFPSGEAQEQLIRSLYQPGGVAPESLEYIEAHGTGTKVGDPQELNGITRSLCAFRQSPLLIGSTKSNMGHPEPASGLAALTKVLLSLENGVWAPNLHFHNPNPEIPALLDGRLQVVDRPLPVRGGIVGINSFGFGGANVHVILQPNTQQAPAPAPHAALPHLLHASGRTMEAVQGLLEQGRQHSQDLAFVSMLNDIAATPTAAMPFRGYTVLGVEGHVQEVQQVPASQRPLWFICSGMGTQWRGMGLSLMRLDSFRESILRSDEALKPLGVKVSDLLLSTDEHTFDDIVHSFVSLTAIQIALIDLLTSMGLKPDGIIGHSLGEVACGYADGCLSQREAVLAAYWRGQCIKDANLPAGSMAAVGLSWEECKQRCPPGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHVPEHAVVLEIAPHALLQAVLKRGVKPSCTIIPLMKRDHKDNLEFFLTNLGKVHLTGIDINPNALFPPVEFPVPRGTPLISPHIKWDHSQTWDIPVAEDFPNGSSSSSATVYNIDASSESSDHYLVDHCIDGRVLFPGTGYLYLVWKTLARSLSLSLEETPVVFENVTFHQATILPRTGTVPLEVRLLEASHAFEVSDSGNLIVSGKVYQWEDPDSKLFDHPEVPIPAESESVSRLTQGEVYKELRLRGYDYGPHFQGVYEATLEGEQGKLLWKDNWVTFMDTMLQISILGFSKQSLQLPTRVTAIYIDPATHLQKVYMLEGDTQVADVTTSRCLGVTVSGGVYISRLQTTATSRRQQEQLVPTLEKFVFTPHVEPECLSESAILQKELQLCKGLAKALQTKATQQGLKMTVPGLEDLPQHGLPRLLAAACQLQLNGNLQLELGEVLARERLLLPEDPLISGLLNSQALKACIDTALENLSTLKMKVVEVLAGEGHLYSHISALLNTQPMLQLEYTATDRHPQALKDVQTKLQQHDVAQGQWDPSGPAPTNLGALDLVVCNCALATLGDPALALDNMVAALKDGGFLLMHTVLKGHALGETLACLPSEVQPGPSFLSQEEWESLFSRKALHLVGLKKSFYGTALFLCRRLSPQDKPIFLPVEDTSFQWVDSLKSILATSSSQPVWLTAMNCPTSGVVGLVNCLRKEPGGHRIRCILLSNLSSTSHVPKLDPGSSELQKVLESDLVMNVYRDGAWGAFRHFQLEQDKPEEQTAHAFVNVLTRGDLASIRWVSSPLKHMQPPSSSGAQLCTVYYASLNFRDIMLATGKLSPDAIPGKWASRDCMLGMEFSGRDKCGRRVMGLVPAEGLATSVLLSPDFLWDVPSSWTLEEAASVPVVYTTAYYSLVVRGRIQHGETVLIHSGSGGVGQAAISIALSLGCRVFTTVGSAEKRAYLQARFPQLDDTSFANSRDTSFEQHVLLHTGGKGVDLVLNSLAEEKLQASVRCLAQHGRFLEIGKFDLSNNHPLGMAIFLKNVTFHGILLDALFEGANDSWREVAELLKAGIRDGVVKPLKCTVFPKAQVEDAFRYMAQGKHIGKVLVQVREEEPEAMLPGAQPTLISAISKTFCPEHKSYIITGGLGGFGLELARWLVLRGAQRLVLTSRSGIRTGYQAKHVREWRRQGIHVLVSTSNVSSLEGARALIAEATKLGPVGGVFNLAMVLRDAMLENQTPELFQDVNKPKYNGTLNLDRATREACPELDYFVAFSSVSCGRGNAGQSNYGFANSTMERICEQRRHDGLPGLAVQWGAIGDVGIILEAMGTNDTVVGGTLPQRISSCMEVLDLFLNQPHAVLSSFVLVEKKAVAHGDGEAQRDLVKAVAHILGIRDLAGINLDSSLADLGLDSLMGVEVRQILEREHDLVLPIREVRQLTLRKLQEMSSKAGSDTELAAPKSKNDTSLKQAQLNLSILLVNPEGPTLTRLNSVQSSERPLFLVHPIEGSITVFHSLAAKLSVPTYGLQCTQAAPLDSIPNLAYTQSYRAKLTPGCEAEAEAEAICFFIKQFVDAEHSKVLEALLPLKSLEDRVAAAVDLITRSHQSLDRRDLSFAAVSFYYKLRAADQYKPKAKYHGNVILLRAKTGGTYGEDLGADYNLSQVCDGKVSVHIIEGDHRTLLEGRGLESIINIIHSSLAEPRVSVRE

SEQ ID NO: 2 - protein, 263 aa, Rat
METAVNAKSPRNEKVLNCLYQNPDAVFKLICFPWAGGGSIHFAKWGQKINDSLEVHAVRLAGRETRLGEPFANDIYQIADEIVTALLPIIQDKAFAFFGHSFGSYIALITALLLKEKYKMEPLHIFVSGASAPHSTSRPQVPDLNELTEEQVRHHLLDFGGTPKHLIEDQDVLRMFIPLLKADAGVVKKFIFDKPSKALLSLDITGFLGSEDTIKDIEGWQDLTSGKFDVHMLPGDHFYLMKPDNENFIKNYIAKCLELSSLT

SEQ ID NO: 3 - protein, 309 aa, Human
MVFPAKRFCLVPSMEGVRWAFSCGTWLPSRAEWLLAVRSIQPEEKERIGQFVFARDAKAAMAGRLMIRKLVAEKLNIPWNHIRLQRTAKGKPVLAKDSSNPYPNFNFNISHQGDYAVLAAEPELQVGIDIMKTSFPGRGSIPEFFHIMKRKFTNKEWETIRSFKDEWTQLDMFYRNWALKESFIKAIGVGLGFELQRLEFDLSPLNLDIGQVYKETRLFLDGEEEKEWAFEESKIDEHHFVAVALRKPDGSRHQDVPSQDDSKPTQRQFTILNFNDLMSSAVPMTPEDPSFWDCFCFTEEIPIRNGTKS

(19) United States
(12) Patent Application Publication
(10) Pub. No.: US 2009/0209015 A1
(43) Pub. Date: Aug. 20, 2009

(54) COMPOSITIONS AND METHODS FOR PRODUCTION OF BIOFUELS

(76) Inventors: Chakkodabylu S. Ramesha, San Jose, CA (US); Anil K. Joshi, North Potomac, MD (US); Raj Subramaniam, Fremont, CA (US)

Correspondence Address: HODGSON RUSS LLP;THE GUARANTY BUILDING, 140 PEARL STREET, SUITE 100, BUFFALO, NY, 14202-4040, US

(21) Appl. No.: 12/372,465

(22) Filed: Feb. 17, 2009

Related U.S. Application Data
(60) Provisional application No. 61/065,851, filed on Feb. 15, 2008.

(51) Int. Cl. C12P 7/64

(52) U.S. Cl. 435/134

(57) ABSTRACT

Provided is a method for producing C4 to C14 fatty acids. The method entails providing genetically modified cells that express a heterologous animal TE-II and either a heterologous animal wild type FAS or a ΔTE-I-FAS. The TE-II and the heterologous wild-type or ΔTE-I-FAS are expressed as distinct molecules. The cells are cultured for a period of time, and the fatty acids are extracted either as free fatty acids or as glycerides from the cells and/or from the media in which the cells are cultured.

This application claims priority to U.S. patent application No. 61/065,851, filed Feb. 15, 2008, the disclosure of which is incorporated herein by reference.

### FIELD OF THE INVENTION

The present invention relates generally biofuels, and more specifically to production of fatty acids for use as biofuels using genetically modified organisms.

### BACKGROUND OF THE INVENTION

The search for biofuels as an alternate energy source is rapidly growing. Ethanol and plant derived oils are the two predominant areas of research and development in the biofuel area. Currently, most of the world's ethanol is produced by bacterial or yeast fermentation of cornstarch, but the direct and indirect costs that go into the production of ethanol barely offset the gain from its use as an alternative fuel. With respect to plant derived oils, recombinant technology has been widely used for the enhancement of oil production in higher plants. However, because of the considerable amount of land and resources required to maintain high-density plants, and because of their slow growth rate, the cost of producing biofuels using land plants in their current form is expected to be high.

The principle ingredients in plant derived oils (as well as in animal fat) are hydrocarbon chain fatty acids which often exist as glycerides or waxes. These fatty acids are synthesized in vivo from 2-carbon units (acetyl- and melonyl-CoA) by the enzyme system termed Fatty Acid Synthase (FAS). Fatty acid synthesis in plants and prokaryotes involves coordinated functioning of seven individual proteins to form a functional FAS complex (1-3). Simultaneous manipulation of all the seven genes responsible for these proteins is necessary to modulate fatty acid content in the transgenic plants. However, in contrast to prokaryotes and plants, animal FAS is a multi-domain homodimeric protein that catalyzes all the steps in fatty acid synthesis. It utilizes acetyl CoA, malonyl CoA and NADPH as substrates to catalyze sequential reactions resulting in the formation of predominantly C16 palmitic acid. The carbon chain length of fatty acids released form the active site of animal FAS is determined by thioesterase activity provided by a thioesterase domain (TE-I). Specifically, TE-I activity causes release of fatty acids when the fatty acid chain length reaches >C12, and results in the predominant production of C16 and C18 fatty acids. However, some tissues naturally make short chain fatty acids via the activity of a separate enzyme (thioesterase-II, TE-II). For example, the cells in lactating mammary glands and uropygial glands in birds express a distinct TE-II protein along with FAS (7, 8). The TE-II protein causes early chain termination resulting in the synthesis of predominantly medium-chain (C8-C12) fatty acids in these tissues. Fatty acids, and in particular short and medium (C6-C12) chain fatty acids, are desirable because of their physical properties and relatively higher caloric values and they can be used directly as biofuels. In addition, they can also be converted to their corresponding alcohols, such as hexanol and/or octanol, which are preferable for many direct commercial uses.

Over-expression of endogenous or animal FAS in E. coli, yeast, and other microbial as well as animal systems has been previously reported (4-6). However, the non-microbial systems are too costly for production of biofuels, and the reported activities of recombinant FAS proteins purified from microbial systems are very low. Recombinant technology has also been used to express fusion proteins for fatty acid synthesis. For example, Joshi, et. al., (9) disclosed expression of two chimeric fusion proteins. Each chimeric protein contained a mammalian FAS having TE-I domain deleted and replaced with a TE-II amino acid sequence. The two chimeric proteins differed from each other by the sequence and length of a linker region between the FAS ACP (acyl carrier protein) domain and TE-II amino acid sequences. Each chimeric protein was separately expressed in and purified from insect cell cultures and fatty acids were synthesized in cell free systems using the purified chimeric proteins. The fatty acid synthesis activity of the purified chimeric proteins was less than 10% of the purified recombinant rat wild type FAS. Organisms expressing either of these two fusion proteins are not considered viable tools for synthesis of fatty acids for use as biofuels. Thus, there is an ongoing need for efficient systems and methods for producing biofuels.

### SUMMARY OF THE INVENTION

The present invention is based on the discovery that genetically modified cells that express as distinct molecules, a heterologous animal TE-II and a heterologous wild type animal FAS, can be used to synthesize fatty acids that are enriched for C14 and shorter carbon chain fatty acids. The genetically modified cells can also express a distinct heterologous animal FAS lacking a TE-I domain (ΔTE-I-FAS), instead of a wild type animal FAS. Accordingly, the present invention provides methods for producing fatty acid compositions enriched in C4 to C14 acids from genetically modified cells. The fatty acids produced by the genetically modified cells can be free fatty acids, or esterified fatty acids, such as glycerides. The glycerides can be monoglycerides, diglycerides, or triglycerides, and can be in the form of phosphoglycerides.

In the method of the invention, the genetically modified cells are maintained in a suitable media for a period of time to allow cells to produce fatty acids. Such fatty acids include C4-C14 fatty acids. The fatty acids produced by the genetically modified cells provided herein contain a higher percentage of C4 to C14 fatty acids than control cells. The fatty acids produced by the cells can be extracted from the cells and/or from the media and are suitable for use as biofuels. The extracted fatty acids can optionally be reduced to their corresponding alcohols, such as butanol, hexanol, octanol, decanol, dodecanol or tertadecanol.

The animal FAS and TE-II enzymes used in the method can be expressed from genes obtained from any animal. In one embodiment, the FAS and TE-II enzymes are expressed from rat genes. The ΔTE-I-FAS enzyme can also be derived from a wild type FAS expressed by any animal.

The invention also provides the genetically modified cells that are suitable for use in the method of the invention. These cells express a heterologous animal TE-II. If the cells do not normally express an endogenous animal FAS, then in addition to the heterologous animal TE-II, they also express as a distinct molecule either a heterologous wild type animal FAS or an animal ΔTE-I-FAS. The heterologous TE-II and FAS or ΔTE-I-FAS genes can be present in the genetically modified cells as DNA polynucleotides that are transiently or stably inserted into the cells.

The cells utilized in the invention can be prokaryotic or eukaryotic cells. In various non-limiting embodiments, the cells can be animal cells, bacteria cells, yeasts, or algae cells. The method is suitable for scaling such that large amounts of fatty acids can be produced for a variety of commercial applications, such as powering combustion engines, turbines, and the like.

### BRIEF DESCRIPTION OF THE FIGURES

FIG. 1 provides the amino acid sequence of rat fatty acid synthase (FAS) protein (SEQ ID NO:1). As shown in FIG. 1, a TE-I deleted FAS (ΔTEI-FAS) protein can be constructed by deleting the sequence downstream of K₂₁₉₉ and engineering a stop codon (⇑) after K₂₁₉₉.

FIG. 2 provides the amino acid sequence of rat thioesterase-II (TE-II) protein (SEQ ID NO:2).

FIG. 3 provides the amino acid sequence of human phosphopantetheinyl transferase (PPTase); (SEQ ID NO:3).

### DESCRIPTION OF THE INVENTION

The present invention provides genetically modified cells that produce an altered fatty acid profile such that C4-C14 fatty acids make up a higher percentage of the total fatty acids, as compared to fatty acids made by cells without the genetic modification. Since C4-C14 fatty acids are suitable for use as fuels directly or via conversion to another fuel, the genetically modified cells provide an efficient source of biofuels.

The fatty acids produced by the genetically modified cells can be free fatty acids, or they may be in the form of esterified fatty acids, such as glycerides. Glycerides can be monoglycerides, diglycerides, or triglycerides, and can be in the form of phosphoglycerides. Fatty acids released from cells, such as by secretion, are predominantly free fatty acids, while cell-associated fatty acids are free fatty acids, as well as esterified forms of fatty acids. Both free fatty acids and esterified fatty acids can be used as biofuels.

The invention provides genetically modified cells that have been engineered to express heterologous proteins that are involved in fatty acid synthesis. By “heterologous” it is meant that the protein is one that is not normally encoded by the particular cell type or organism in which the protein is expressed for use in the invention.

The cells used in the invention are engineered to express a heterologous animal TE-II. While heterologous TE-II can be used as the only inserted heterologous gene to achieve an alteration in fatty acid profile produced by the cells, it is preferred that the cells are genetically modified to express the heterologous TE-II in combination with a distinct heterologous wild-type or modified animal FAS. The modified animal FAS can be an animal FAS that has been modified so that it lacks a TE-I domain (ΔTE-I-FAS).

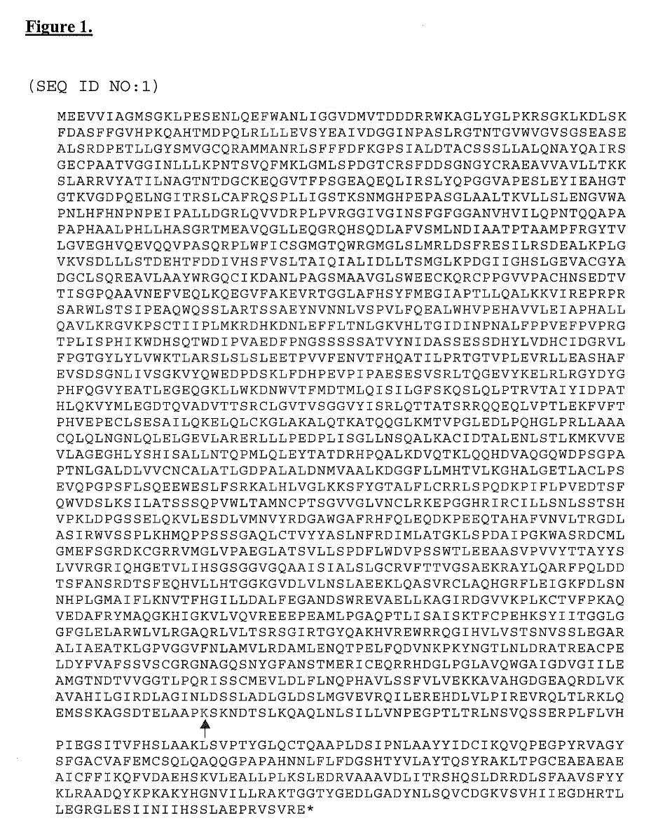

The amino acid sequences of the heterologous animal proteins used in the method can be the same as those expressed by any animal species, or may comprise experimentally altered amino acid sequences. A variety of wild type animal FAS amino acid sequences are currently known in the art, but it is expected that animal FAS sequences hereafter identified could also be used. Some non-limiting examples of FAS proteins suitable for use in the invention include rat, chicken, human, mouse and cow FAS. The amino acid sequence of rat FAS is described in Amy et al., Proc. Natl. Acad. Sci. U.S.A. 86 (9), 3114-3118 (1989) and is also provided as NCBI Accession No. AAA41145 (Apr. 27, 1993 entry). An annotated rat FAS sequence is also provided in FIG. 1. The amino acid sequence of FAS from chicken (Gallus gallus) is provided under NCBI Accession No. J03860 (Apr. 28, 1993 entry). The amino acid sequence of human FAS is provided under NCBI Accession No. NM_{—}004104 (Dec. 21, 2003 entry). The amino acid sequence of mouse (Mus musculus) FAS is provided under NCBI Accession No M_{—}007988 (Mar. 5, 2006 entry). The amino acid sequence of the domestic cow (Bos Taurus) FAS is provided under NCBI Accession No AY343889 (Aug. 25, 2005 entry). The mRNA and deduced amino acid sequence of porcine FAS is also known (J. Nutr. 121, 900-907, 1991).

Animal FAS is a well characterized protein that contains seven catalytic domains. The TE-I domain is the most C-terminal of the seven. While FAS amino acid sequences may differ somewhat between animal species, each of the seven domains in any particular FAS amino acid sequence can be readily recognized by those skilled in the art. Accordingly, any TE-I domain can be identified by one skilled in the art and removed from any FAS using standard molecular biology techniques to thereby obtain a ΔTE-I-FAS. An exemplary ΔTE-I-FAS amino acid sequence is provided in FIG. 1 (SEQ ID NO:1) which is expressed from a coding sequence having an engineered stop codon after the codon for K₂₁₉₉. Thus, the sequence downstream (C-terminal) of K₂₁₉₉, which constitutes the TE-I domain, is not present in the ΔTE-I-FAS. Accordingly, amino acids 1 through 2199 shown in FIG. 1 illustrates the amino acid sequence of an exemplary ΔTE-I-FAS derived from rat FAS.

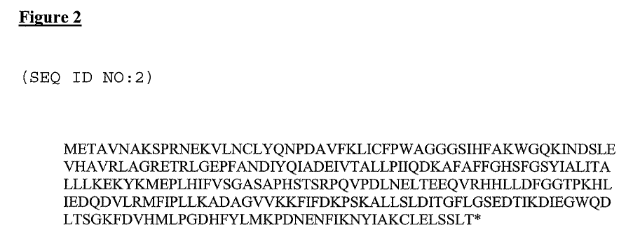

As in the case for FAS, a variety of animal TE-II amino acid sequences are currently known in the art. One example of a suitable TE-II amino acid sequence is provided in FIG. 2 (SEQ ID NO:2) which shows rat TE-II, the sequence of which was described by Naggert et. al. (Biochem. J. vol. 243, pp. 597-601, 1987). The sequence of TE II from the Uropygial gland of mallard duck is also known (Poulose A J, Rogers L, Cheesbrough T M, Kolattukudy P E. J. Biol. Chem. vol. 260(29), pp. 15953-8, 1985). TE-II from other animal sources could also be used in the present invention.

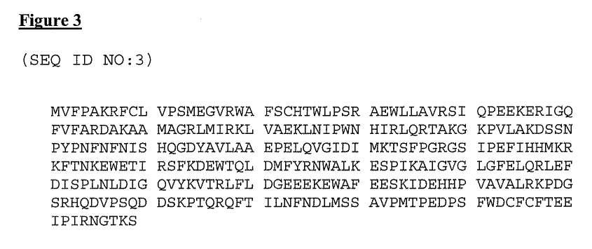

In another embodiment, in addition to animal ΔTE-I-FAS and TE-II, the invention provides for the optional expression of a heterologous wild type or mutated PPTase gene so as to increase fatty acid synthesis. In this regard, several PPTase enzymes are known to pantetheinylate apo-FAS to generate active holo-FAS. In one embodiment, the PPTase enzyme may have the same sequence as a human PPTase enzyme. The amino acid sequence for a human PPTase enzyme is provided in FIG. 3 (SEQ ID NO:3).

For the animal enzymes utilized in the present invention, it is expected that either wild type amino acid sequences, or sequences having homology with the wild type amino acid sequences could be used. In this regard, for proteins that comprise amino acid sequences that are not identical to the wild type sequences, it is considered that proteins that have at least 80% homology, and more preferably 90%, 95%, or 99% homology with the wild type sequences, could be used in the invention. Due to the well characterized nature of wild type animal FAS and TE-II proteins, those skilled in the art can determine which amino acid positions can tolerate substitutions, and what amino acid substitutions could be made at those positions using routine experimentation.

While specific amino acid sequences for examples of enzymes that can be used in the present invention are provided herein, it will be recognized that there are a multitude of DNA sequences that can encode such amino acid sequences due to the redundancy of the genetic code. Thus, cells comprising any DNA sequences encoding these enzymes are included within the scope of the invention. Further, cells comprising intronic sequences of the mammalian genes are also included, should the enzyme coding sequences used in the invention be cloned directly from mammalian genomic DNA.

The heterologous TE-II and FAS or ΔTE-I-FAS genes can be inserted into and expressed by the genetically modified cells using any suitable reagents and techniques. In general, the heterologous genes are expressed from DNA polynucleotides that have been transiently or stably inserted into the cells. For instance, for transient insertions, DNA polynucleotides encoding the heterologous genes can be expressed from one or more expression vectors that are inserted into the cells and are replicated independently of the host DNA. Suitable expression vectors can be obtained from commercial vendors or constructed in the laboratory such that the co-expression of multiple genes of interest in a desired cell type can be achieved. Such vectors can include various promoters and other regulatory elements to effect and/or optimize expression of the inserted genes. For instance, suitable expression vectors may comprise prokaryotic and/or eukaryotic promoters, enhancer elements, and selectable markers for use in maintaining one or more distinct expression vectors in the desired cell type. Some representative commercially available systems include but are not limited to, for insect cell expression: pFastBac vectors and Bac-to-Bac System (available from INVITROGEN®); for E. coli expression: pET vectors (available from NOVAGEN®) and pQE vectors (available from QIAGEN®); for S. cervisiae expression: pYES2-DEST52 expression vector (available from INVITROGEN®); for Pichia pastoris expression: Easy Select™ expression kit (available from INVITOGEN®), and for algae, AlgRx™ Algal Protein Expression System (available from Rincon Pharmaceuticals.)

In one embodiment, to co-express the inserted genes, the TE-II and either the FAS or the ΔTE-I-FAS and may be encoded by a single expression vector under operative control of a single or distinct promoters, which may be constitutive or inducible promoters. In the case of a single promoter, second or additional genes may be translated from, for example, an internal ribosomal entry sequence(s) from a single mRNA. Alternatively, the polynucleotide sequences encoding the enzymes may be expressed in any single cell by distinct expression vectors which may also comprise any of a wide variety of constitutive or inducible promoters, selective markers, etc.

As an alternative to co-expression of distinct proteins, a novel fusion protein provided by the invention may be translated from a single open reading frame on an mRNA, the transcription of which is driven from any of a wide variety of promoters in any suitable expression vector. For example, the invention contemplates providing a novel chimeric animal Δ-TE-I-FAS-TE-II fusion protein that comprises a novel linker sequence joining the Δ-TE-I-FAS and the TE-II such that the fatty acid profile shifts towards C4-C14 fatty acid.

DNA polynucleotides encoding the heterologous animal TE-II and FAS or ΔTE-I-FAS genes can also be stably inserted into cells by integration into the host cell DNA. One advantage of integrating the heterologous genes is that stable transformants are produced and are capable of maintaining the heterologous genes when grown in a non-selective medium. The host DNA into which the DNA polynucleotides encoding the heterologous genes are integrated can be nuclear DNA, or organelle DNA.

While the cells used to express the heterologous proteins could be cells of any species or cell type, including prokaryotic and eukaryotic cells, it is preferred to use microbial cells so that the method can be more conveniently utilized for large-scale production of biofuels. For example, it is expected that bacteria could be used to harness carbon sources, such as from sewage, in the production of fatty acids in the method of the invention. Alternatively, yeast could be used to produce the fatty acids using various fermentation apparatuses.

In one embodiment, the invention utilizes recombinant algae. Algae systems offer an advantage of decreasing the cost of biofuel production because of the rapid growth rates and inexpensive growth conditions, owing to the use of light and CO₂ as the main energy source via the photosynthetic pathway. Algae are plant-like organisms without roots, stems or leaves. Algae contain chlorophyll and vary in size from microscopic forms (phytoplankton) to large seaweeds. Their habitat is fresh or salt water, or moist environments. Most algae are eukaryotic (sub-kingdom=phyciobionta), but several (e.g., cyanobacteria and prochlorophyta) are prokaryotic. Algae suitable for use in the present invention encompass both prokaryotic and eukaryotic algae, and are preferably unicellular algae. Unicellular algae are also known as microalgae. Non-limiting examples of algae that can be used in the invention include Chlorella vulgaris, which is known to secrete high levels of free fatty acids into media, and Chlamydomonas reinhardtii.

Without intending to be bound by any particular theory, it is considered that recombinant protein expression in algae is optimal for use in the invention when it takes place in the chloroplast. The heterologous DNA polynucleotides encoding the TE-II and FAS or ΔTE-I-FAS genes can be stably integrated into the chloroplast DNA of the algae by transforming the algae with one or more vectors which comprise the heterologous genes and which are targeted to the chloroplasts. Methods and vectors that can be used for integrating heterologous genes into algae chloroplast DNA by, for example, homologous recombination have been described. (See, for example, Hutchinson, et al., 1996, Chapter 9, Chloroplast transformation. Pgs. 180-196; In: Molecular Genetics of Photosynthesis, Frontiers in Molecular Biology. Anderson B., Salter A H, and Barber J. eds.: Oxford University (Davies et al. (1994) Plant Cell 6:53-63). Further, Fletcher et al. (Adv Exp Med. Biol. (2007) vol. 616, pp. 90-98) disclose optimization of recombinant protein expression in chloroplasts of green algae. In particular, Fletcher et al. describe optimization of recombinant protein expression in Chlamydomonas reinhardtii chloroplasts by employing chloroplast codon bias and manipulation of promoter and untranslated region (UTR) combinations. They also demonstrate that the C. reinhardtii chloroplast is capable of correctly folding and assembling complex mammalian proteins. Other work with algae systems also demonstrates the successful use of these organisms to efficiently express a variety of proteins (i.e., Franklin et al., Curr Opin Plant Biol. (2004) Vol. 2, pp 159-65; Mayfield, et al. PNAS (2003) Vol. 100, pp 438-442; and Heifetz, P. B. (2000) Biochimie Vol. 82, 655-666). Therefore, it is expected that algae will provide a convenient system for expressing functional proteins that catalyze improved synthesis of fatty acids according to the method of the invention.

The expression of a heterologous wild-type or modified animal FAS with a heterologous TE-II is expected to be advantageous in embodiments wherein the cells are non-animal cells, and thus do not normally exhibit endogenous animal FAS activity. For example, and without intending to be bound by any particular theory, since algae do not endogenously express a FAS that would cooperate with a heterologous animal TE-II in synthesis of fatty acids, it is preferable to express in the algae both the heterologous animal TE-II and a heterologous wild-type or modified animal FAS.

In a preferred embodiment, the heterologous TE-II and the heterologous wild-type or modified FAS are each expressed as distinct protein molecules within the cells. Thus, a method of the present invention comprises providing genetically modified cells that express a heterologous TE-II and either a heterologous FAS or a ΔTE-I-FAS, wherein the TE-II and the heterologous wild-type or ΔTE-I-FAS are expressed as distinct molecules, allowing the genetically modified cells to produce fatty acids, and collecting and/or the extracting the C4-C14 fatty acids, or any combination thereof, produced by the cells.

A convenient method of allowing the cells to produce fatty acids is to culture the cells. The amount of time and the type of apparatus in which the cells are cultured will depend on various factors that will be apparent to those skilled in the art, such as the type of cells being used, the type and volume of the culture medium, temperature, nutrient requirements, and the particular procedure that is being utilized to separate the C4-C14 fatty acids from the cells and/or the culture medium.

In one embodiment, recombinant algae according to the invention are grown in vessels suitable for commercial production of fatty acids for use as biofuels. Non-limiting examples of suitable vessels include plastic bags, and larger containers, such as vats, which may be in the form of photobioreactors. Photobioreactors can be industrial-scale culture vessels made of transparent clear materials (e.g., glass, acrylic, polycarbonate, etc) in which algae can grow and proliferate using photosynthesis to produce energy. If desired, the cultures can be mixed by either a pump or air bubbling. Alternative vessels for growing the algae include but are not limited to shallow open ponds, for example, man-made ponds suitable for holding water levels of approximately 15 to 30 cm high. Such ponds can be constructed as a loop in which the algae culture is circulated by, for instance, a paddle-wheel. Fatty acid production in microalgae has been reported at levels of from 1800-2500 gallons of oil/acre/year. It is expected that the present invention will equal or exceed such production levels.

Methods for separating and purifying fatty acids from a wide variety of cell types and cell culture media are well known in the art and can be used in performing the method of the present invention. For example, when the genetically modified cells used in the invention are algae, fatty acids can be extracted from the algae using a conventional oil press. Alternatively, the well known supercritical fluid method can be used to extract fatty acids from the cells/and or media.

If esterified fatty acids are extracted, they can be hydrolyzed (for example, using enzymatic, acid or base hydrolysis) and the free fatty acids can be separated from the glycerol using well known methods. Free fatty acids can optionally be reduced to their corresponding alcohols or hydrocarbons.

The extracted C4-C14 fatty acids and/or their alcohol derivatives can be used for producing energy that can power a machine, such as a combustion engine, a turbine, or any machine capable of being powered by combustion of the fatty acids and/or their corresponding alcohols.

It is generally considered that the invention provides for an increase in total amount (i.e., mass) of short chain fatty acids synthesized by the cells used in the method. However, the invention also facilitates an increase in relative proportion of short chain fatty acids, as compared to the total fatty acids synthesized. The increase in relative proportion of fatty acids can be an increase of from 1% to 10%, inclusive, and all including all integers between 1% and 10%. An increase in relative proportion of total fatty acids can be evidenced by an increase in the proportion of any or all of C4-C14 fatty acids relative to the total fatty acids synthesized. The total fatty acids synthesized can be measured as the sum of the individual percentages of total fatty acids (such as for C4-C18 fatty acids including C4, C6, C8, C10, C12, C14, C16:0, C16:1, C18:0 and C18:1 fatty acids). Other fatty acids that could constitute more than an insignificant amount of total fatty acids can also be measured as necessary.

In various embodiments, the invention can be used to increase the percentage of any or all of C4 to C14 fatty acids by at least 40%, 50%, 100%, 200%, 300%, 400%, and to greater than 1000%, relative to a corresponding fatty acid profile obtained from control cells (i.e., control cells that comprise an empty expression vector or express only ΔTE-I-FAS without TE-II).

In a particular embodiment, the invention provides for increasing the percentage of C8, C10, C12, and C14 fatty acids by at least 40%, 50%, 100%, 200%, 300%, 400%, and to greater than 1000%, relative to corresponding fatty acid profile obtained from control cells (i.e., control cells that comprise an empty expression vector or express only ΔTE-I-FAS without TE-II).

Relative percentages of fatty acids synthesized by cells can be determined using standard methods. For example, total lipids can be extracted from a cell pellet using the well known method of Bligh and Dyer (Bligh, E. G., and Dyer, W. J. (1959) Can. J. Biochem. Physiol, 37, 911-917). Individual fatty acids can be identified using conventional analytical tools, such as mass spectroscopy or gas chromatography. Fatty acid synthesis profiles can be compared to control cells, such as cells of the same type used in the method, but which do not express a heterologous TE-II, and/or do not express either a heterologous FAS or a ΔTE-I-FAS. The actual amount, i.e., the moles, of each particular fatty acid synthesized can also be determined using standard methods, such as by gas chromatography using internal standards.

The following Examples are meant to illustrate but not limit the invention.

### EXAMPLE 1

This Example provides a description of materials and methods used for cloning Flag-wild type rat FAS, His₆-ΔTE I FAS, and His₆-TE II, and co-expression of the proteins in Sf9 cells.

Anti-FLAG M₂ monoclonal antibody was obtained from Eastman Kodak Co. and Anti-H His 6 monoclonal antibody was obtained from CLONTECH Laboratories Inc. (Palo Alto, Calif.). Alkaline phosphatase-conjugated, goat anti-mouse IgG was purchased from Bio-Rad. The BAC-to-BAC Baculoviral expression system was purchased from Invitrogen (Carlsbad, Calif.). Vent polymerase and restriction enzymes were from New England Biolabs (Ipswich, Mass.).

cDNAs encoding Flag/His₆tagged-wild type rat FAS and expression of recombinant proteins in Sf9 cells was performed using known techniques. Briefly, the His₆-tagged ΔTE I-FAS construct was generated by deleting the whole TE I domain coding sequence of a rat wild type FAS construct (summarized in FIG. 1) and substituting the ACP domain (which is the domain immediately preceding the TE-I domain in the C-terminal direction) with PCR amplified ACP domain sequence carrying a C-terminal STOP codon, using standard cloning techniques. The sequence and location of the primers used to amplify rat ACP substitution fragment are described in Table 1. In Table 1, uppercase letters indicate that primer sequences are identical to cDNA sequence, while bases in lowercase are not present in cDNA and were incorporated into the oligomers in order to engineer restriction sites at the ends of amplified fragments. Restriction sites used for cloning are shown in italics and stop codons are shown in bold. T or B in oligomer names indicates sense/antisense oligomer, respectively. The base pair numbers for ΔTE I-FAS primers are according to the rat FAS cDNA sequence published in Amy et al (1989) Proc. Natl. Acad. Sci. USA 86, 3114-3118), while those for TE II primers are according to the rat TE II sequence published in Naggert et. al. (1987) Biochem. J. 243, 597-601.

TABLE 1

| | | |
|---|---|---|
| Primer | Sequence | Location |

ΔTE I-FAS Primers

| | | |
|---|---|---|
| 200T6080 | 5′-catatctaGACAGGGCGACCCGGGAAGCCTGTCCT | 6070-6096 |
| (SEQ ID NO:4) | Xba I Xma I | |
| 1B6665 | 5′-atatgcggccgcatcCTTGGGGGCTGCCAACTCAGTGTCTG | 6653-6678 |
| (SEQ ID NO:5) | Not I | |
| TE II Primers | | |
| TE2T1 | 5′-atgctagcgcgcATGGAGACCGCTGTTAACGCTA | 1-22 |
| (SEQ ID NO:6) | BssH II | |
| TE2B1 | 5′-tgatagcggccgcTCAAGTGAGTGACGAGAGTTCCA | 770-729 |
| (SEQ ID NO:7) | Not I | |

The coding sequence of the rat TE-II was amplified by PCR using TE II carrying plasmid 233.FB as the template DNA and Vent polymerase as described earlier (Joshi, et al. (1993) Biochem. J. vol. 296, pp. 143-149). The sequence and location of the primers used to amplify rat TE-II are described in Table 1. The sense and antisense primers carried BssH2 and NotI restriction enzyme recognition sites that were used to clone the amplified DNA, in frame, down stream of the His₆ recognition sequence modified pFASTBAC vector using standard cloning techniques. The DNA sequence of the cloned TE-II gene was confirmed by DNA sequencing, and used to generate recombinant Baculoviral stocks by a conventional transposition method, using the BAC-to-BAC Baculoviral expression system (Invitrogen) according to the manufacturer's instructions.

All three recombinant viral stocks (TE-II, FAS and ΔTE I-FAS) were titrated before use. For protein expression Sf9 cells (˜1×10⁶ cells/ml grown in EX-CELL 420 serum free media; SAFC Biosciences) were infected with the recombinant viruses (at specified M.O.I.) and cultured for 48 h at 27° C. SDS PAGE and Western blot analysis of cell lysates using tag specific primary antibody confirmed expression of each protein. For co-expression experiments, the cells were infected with various combinations of recombinant viruses for 48 hr, harvested by centrifugation and used for fatty acid analysis.

### EXAMPLE 2

This Example provides demonstrations of elevated synthesis of C14 and shorter fatty acids achieved by the invention using an insect cell/baculovirus system constructed and used to express the proteins as described in Example 1. The fatty acid content summarized in Table 2 and Table 3 below was determined using conventional methods. Briefly, total lipids from pelleted cells were extracted by the method of Bligh and Dyer (Bligh, E. G., and Dyer, W. J. (1959) Can. J. Biochem. Physiol, 37, 911-917). Esterified fatty acids in the total lipid extract were hydrolyzed using 0.2M KOH, and the free fatty acids were derivatized to pentafluorobenzyl esters and analyzed by gas chromatography-mass spectroscopy (GC-MS) as described by Ramesha and Taylor (Ramesha, C. S., and Taylor, L. A. (1991) Analytical Biochem. 192, 173-190). Individual fatty acids were identified using standards and mass spectra. The concentration of each fatty acid was expressed as the percent of the total fatty acids. Fatty acids longer than C18 were less than 0.02% of the total under the culture conditions and were ignored. The results of fatty acid analysis are as provided in Table 2 and Table 3. Parenthesis indicates multiplicity of infection (M.O.I.).

TABLE 2

| | | Control-2 | | TE-II + | TE-II + | TE-II + | | |
|---|---|---|---|---|---|---|---|---|
| | Control-1 | (ΔTE- | TE-II | ΔTE-I- | ΔTE-I- | ΔTE-I- | TE-II + | TE-II + |
| | (empty | I-FAS | only | FAS | FAS | FAS | FAS | FAS |
| | vector) | alone) | (100)^{a} | (100 + 100) | (25 + 50) | (25 + 100) | (25 + 50) | (25 + 100) |
| C8 | ND* | 0.001 | 0.46 | 0.61 | 0.084 | 0.184 | 0.442 | 0.156 |
| C10 | ND | 0.074 | 0.89 | 0.6 | 0.381 | 0.412 | 0.594 | 0.378 |
| C12 | 0.59 | 0.489 | 5.9 | 3.62 | 2.302 | 2.425 | 2.424 | 2.235 |
| C14 | ND | 0.312 | 4.02 | 2.95 | 0.874 | 0.883 | 0.928 | 0.859 |
| C16:1 | 27.3 | 28.992 | 22.47 | 24.01 | 26.489 | 25.932 | 27.343 | 27.389 |
| C16:0 | 5.07 | 4.604 | 5.54 | 5.26 | 5.473 | 5.261 | 5.756 | 5.393 |
| C18:1 | 49.6 | 52.3 | 45.47 | 48.39 | 48.599 | 49.441 | 46.663 | 47.355 |
| C18:0 | 16.9 | 13.229 | 14.75 | 14.1 | 15.797 | 15.462 | 15.851 | 16.236 |
| A | 0.59 | 0.876 | 11.27 | 7.78 | 3.641 | 3.904 | 4.388 | 3.628 |
| B | 0.733 | | | | | | | |

A: % increase in C8 to C14 fatty acids
B: Average % increase of control 1 and control 2

TABLE 3

| | | TE-II + | TE-II + | TE-II + | | |
|---|---|---|---|---|---|---|
| | | ΔTE-I- | ΔTE-I- | ΔTE-I- | TE-II + | TE-II + |
| | TE-II | FAS | FAS | FAS | FAS | FAS |
| Control | (100) | (100:100) | (25:50) | (25:100) | (25:50) | (25:100) |
| 100% | 1538% | 1061% | 497% | 533% | 599% | 495% |

Table 3 provides a summary of % increase in C8 to C14 relative to the average of the percent increase in C8:0 to C14:0 for the two controls. The average of the two controls (0.733% increase) is set to 100%, as shown for “Control” in Table 3.

It can be seen from Tables 2 and 3 that the control insect cells contain very low levels of C8 to C14 totaling <1% of the total fatty acids. However, expression of TE-II alone in these cells results in early chain termination of the native (endogenous) FAS, thus resulting in a higher abundance of shorter chain fatty acids, totaling about 10%. Co-expression of both ΔTE-I-FAS and TE-II also results in early termination of fatty acid chain resulting in higher abundance of shorter chain fatty acids (up to ˜10%). In addition to the early chain termination of fatty acids in TE-II alone and TE-II+ΔTE-I-FAS expressing cells, cells expressing wild type FAS along with TE-II also show early chain termination.

Although the total amounts of C8-C14 fatty acids produced are less than 15% of the total fatty acids, the absolute increase in the short chain fatty acids (C8 to C14) represents from 400 to >1000% increase over the control cells (Table 3).

For the data presented in Tables 2-3, pre-existing and newly synthesized fatty acids following expression of the recombinant proteins were not distinguished. Also, the fatty acids accumulation occurred for only about 36 hrs during the 48 hr infection period. Therefore, it is likely that the shorter chain fatty acids represent a far greater proportion of the newly synthesized fatty acids than shown in the Tables. Furthermore, it is likely that C4 and C6 fatty acids are also formed following the expression of TE-II and ΔTE-I-FAS and TE-II, even though they were not measured in this particular case.

Thus, it is clear from the above data that, by expression of a heterologous animal TE-II alone or such a TE-II in association with a heterologous wild type or modified FAS, it is possible to increase the proportion of short C4-C14 fatty acids synthesized by the cells, relative to total fatty acid synthesis. It is expected that the method of the invention could be performed in any suitable cell type with similar results, and indicates that the invention will be useful for large scale production of short chain fatty acids suitable for use as biofuels.

### REFERENCES

1. D. G. Hardie, & A. D. McCarthy (1986) in Mutidomain Proteins-Structure and Evolution, eds. Hardie, D. G. & Coggins, J. R. (Elsevier, Amsterdam), pp. 229-258.
2. S. Smith (2006) Science, Vol. 311, pp 1251-1252
3. S. Smith, FASEB J. (1994) vol. 8, pp. 1248-59
4. E. Schweizer and J. Hofmann (2004) Microbiology and Molecular Biology Reviews, Vil. 68, pp. 501-517 and references therein
5. V. Jayakumar, W-Y Huang, B. Raetz, S. S. Chirala, and S. J. Wakil (1996) Proc. Natl. Acad. Sci. USA. Vol. 93, pp. 14509-14514.
6. A. K. Joshi, S. Smith (1993) Biochem. J. vol. 296, pp. 143-149.
7. L. Libertini, C-Y. Lin, and S. Smith, (1976) Fed. Proc. 35.1671 and references therein.
8. J. S. Buckner, and P. E. Kolattukudy, (1976) in Chemistry and Biochemistry of Natural Waxes (Kolattukudy, P. E., ed) pp. 147-200, Elsevier, New York
9. A. K. Joshi, et al (2005) Biochemistry, Vol. 44, pp 4100-4107
10. M. Kates, (1986) Techniques of Lipidology, 2^{nd} Revised Edition, Elsevier Publications.

We claim:

1. A method for producing fatty acids comprising the steps of:
providing genetically modified cells that express as distinct molecules:
a) a heterologous animal thioesterase-II (TE-II), and
b) a heterologous animal fatty acid synthase (FAS) or a heterologous animal FAS lacking a thioesterase I (TE-I) domain,
maintaining the cells in a media;
collecting the cells and/or the media; and
extracting the fatty acids from the genetically modified cells and/or the media.

2. The method of claim 1, wherein the fatty acids extracted from the cells and/or the media comprise C4-C14 fatty acids as free fatty acids, as esterified fatty acids, or a combination of free fatty acids and esterified fatty acids.

3. The method of claim 2, wherein an amount of any of C4-C14 fatty acids extracted from the cells and/or the media is a greater percentage of total fatty acids as compared to C4-C14 fatty acids measured as a percentage of total fatty acids produced by control cells that do not comprise a heterologous animal TE-II, a heterologous animal FAS, or a FAS lacking a TE-I domain.

4. The method of claim 1, wherein the fatty acids extracted from the cells and/or the media comprise C4-C14 fatty acids, and wherein the C4-C14 fatty acids are at least 5% of total fatty acids extracted from the cells and/or the media.

5. The method of claim 4, wherein the fatty acids extracted from the cells and/or the media comprise C4-C14 fatty acids, and wherein the C4-C14 fatty acids are at least 10% of total fatty acids extracted from the cells and/or the media.

6. The method of claim 1, further comprising converting one or more of the fatty acids extracted from the cells and/or the media to an alcohol.

7. The method of claim 1, further comprising combusting the fatty acids extracted from the cells and/or the media to provide power to a machine.

8. The method of claim 7, further comprising combusting the alcohol to provide power to a machine.

9. The method of claim 1, wherein the cells express the heterologous TE-II and the FAS lacking a TE-I domain, but not the heterologous FAS.

10. The method of claim 1, wherein the cells express the heterologous TE-II and the heterologous FAS, but not the FAS lacking a TE-I domain.

11. The method of claim 1, wherein the genetically modified cells comprise DNA sequences encoding the heterologous TE-II and the heterologous animal FAS or the heterologous animal FAS lacking a TE-I domain on an expression vector.

12. The method of claim 1, wherein the genetically modified cells comprise DNA sequences encoding the heterologous TE-II and the heterologous animal FAS or the heterologous animal FAS lacking a TE-I domain integrated into nuclear DNA or organelle DNA of the genetically modified cells.

13. The method of claim 1, wherein the genetically modified cells are eukaryotic cells.

14. The method of claim 1, wherein the genetically modified cells are prokaryotic cells.

15. The method of claim 13, wherein the eukaryotic cells are yeast cells.

16. The method of claim 14, wherein the prokaryotic cells are bacteria.

17. The method of claim 1, wherein the genetically modified cells are algae.

18. The method of claim 17, wherein the algae comprise DNA sequences encoding the heterologous TE-II and the heterologous animal FAS or the heterologous animal FAS lacking a TE-I integrated into chloroplast DNA.

19. The method of claim 17, wherein the algae are maintained in the media in a photobioreactor.

20. The method of claim 17, wherein the algae are maintained in the media in a pond.
